(19) United States
(12) Patent Application Publication
(10) Pub. No.: US 2026/0224234 A1
(43) Pub. Date: Aug. 6, 2026

(54) BALLOON CATHETER-ASSISTED TRAVERSAL OF BLOOD VESSEL OBSTRUCTIONS

(71) Applicant: Timothy Patrick Murphy, Somerville, MA (US)

(72) Inventor: Timothy Patrick Murphy, Somerville, MA (US)

(21) Appl. No.: 19/538,442

(22) Filed: Feb. 12, 2026

Related U.S. Application Data
(63) Continuation-in-part of application No. 18/106,262, filed on Feb. 6, 2023, now Pat. No. 12,551,668.
(63) Continuation-in-part of application No. 19/384,183, filed on Nov. 10, 2025.

(51) Int. Cl. A61B 17/22; A61B 17/00

(52) U.S. Cl. CPC A61B 17/22; A61B 17/00234; A61B 2017/00305; A61B 2017/00778; A61B 2017/22001; A61B 2017/22038; A61B 2017/22051; A61B 2017/22094

(57) ABSTRACT

The disclosed invention is in the field of medical procedure, namely catheter-based procedures to achieve traversal of obstructions in blood vessels. The method comprises use of a balloon catheter, and inflation of a balloon within an obstruction in a blood vessel during guidewire and catheter traversal is used to achieve space for advancement of a guidewire.

### CROSS-REFERENCE TO RELATED APPLICATIONS

This application is a continuation-in-part of U.S. application Ser. No. 18/106,262, filed Feb. 6, 2023, entitled “Angioplasty Balloon Catheter Comprising a Biaxial-Coaxial Transition”, Inventor Timothy Murphy, and claims the benefit thereof under 35 U.S.C. § 120 and 37 C.F.R. § 1.78.

This application is a continuation-in-part of U.S. application Ser. No. 19/384,183, filed Nov. 10, 2025, entitled “Injectable Angioplasty Balloon Catheter Comprising a Biaxial-Coaxial Transition”, Inventor Timothy Murphy, and claims the benefit thereof under 35 U.S.C. § 120 and 37 C.F.R. § 1.78.

### FIELD OF THE INVENTION

The present invention relates to medical devices and medical vascular interventional methods. More particularly, the present invention is directed to medical methods used for treatment of blockages in blood vessels.

### BACKGROUND

This description of art is not intended to constitute an admission that any patent, publication, or other information referenced is “prior art” with respect to the invention unless specifically designated as such. In addition, this section should not be construed to mean that a search has been made or that no other pertinent information as defined in 37 C.F.R. § 1.56(a) exists.

Balloon catheters have been used to dilate blockages in blood vessels for over 40 years. An balloon catheter comprises a tubular element with an expansile member generally disposed toward a distal end.

Angioplasty procedures are typically performed using percutaneous access to a blood vessel by the Seldinger technique. The Seldinger technique involves placement of a needle through the skin into a blood vessel, passage of a guide wire through a lumen of the needle into the blood vessel, and removal of the needle and replacement with a plastic diagnostic catheter. Once Seldinger access of a catheter into a blood vessel is achieved, the guide wire and catheter combination are used by an operator to selectively catheterize a blood vessel with a blockage to be treated, arteriograms are obtained, and then a balloon catheter is substituted for the diagnostic catheter and positioned so that an inflatable balloon disposed toward a distal tip of said balloon catheter is generally positioned centrally within said blockage. Said balloon is then expanded by injection of fluids into a lumen in communication with an interior of said balloon, relieving the said blockage. Said balloon is then deflated by aspiration of said injected fluids, and the said balloon catheter is removed from the body.

Lesion traversal is a key step in any catheter revascularization procedure. When obstructions in arteries are severe or completely occluded, traversal with conventional guidewires and catheters can be challenging. The goal is to cross the blockage in a blood vessel and achieve guidewire and catheter position within a downstream unobstructed segment. Typically, attempts are made to cross blockages with a combination of a standard diagnostic catheter and a guidewire. Alternatively, specialized “crossing catheters” are often used to traverse such obstructions and then are exchanged for other revascularization catheters such as angioplasty balloon catheters or atherectomy catheters that cut away obstructions. These crossing catheters often have low profile tip entry characteristics and flexible tips to provide “trackability”, hydrophilic coatings, and a robust proximal shaft to provide column strength or “pushability”. Some examples of crossing catheters include the QuickCross Catheter (Philips Healthcare, Inc., Amsterdam, Netherlands) and CXi (Cook Medical, Inc., Bloomington, IN, USA). Other crossing catheters have needles (Outback, Cordis, Miami Lakes, FL, USA) or RF energy (PowerWireR, Bayliss Medical Technologies, Mississauga, ON, Canada) to assist with crossing obstructions in blood vessels. Typically, low profile crossing catheters are introduced over 0.014″ or 0.018″ outer diameter (OD) guidewires.

### SUMMARY

In accordance with one form of the invention, a method of treatment of an obstruction in a blood vessel is disclosed that comprises crossing an obstruction in a blood vessel by use of a catheter with an expansile member or balloon disposed adjacent to its distal tip, and inflating said expansile member to bluntly dissect the interior of said obstruction to enable traversal of a guidewire through it.

In some embodiments said balloon catheter may comprise a feature of injectability while in an over-the-wire configuration, viz, with a guidewire residing in situ throughout most or all of a length of an inner lumen of said balloon catheter. For the purposes of illustration, such a catheter shall be termed an “injectable balloon catheter.” Injectability while over a guidewire is desirable because it allows an operator to inject radiopaque contrast and thereby visualize their progress while attempting to traverse an obstruction in a blood vessel. While all over the wire catheters are injectable when a guidewire is not present in their lumens, a more useful embodiment is to allow catheter injection while over a guidewire, what we term “injectability”. Said injection may be accomplished using injection exit ports proximal to an balloon, or distal to a balloon, the latter including from the distal tip. Said injectability may be provided with said catheter is in an over-the-guidewire configuration. When injection is desired from a distal tip said guidewire may be retracted into a larger proximal lumen segment that allows contrast to flow around said guidewire but then out said distal exit hole when said guidewire is retracted proximally. That maneuver is useful in determining the anatomy forward of the catheter distal tip. Specifically, injections of contrast that exit the distal tip during crossing of a blood vessel obstruction are useful to confirm whether or not the catheter remains in the desired lumen of the blood vessel, or whether it has attained an intramural path within the blood vessel wall, or whether it has perforate to a location external to a blood vessel.

One exemplary way of performing a revascularization procedure of an occluded blood vessel considers the example of an athersclerotic obstruction of an artery. A catheter and guidewire are introduced into the body of a patient and then direct by an operator, typically with fluoroscopic guidance, to the proximal end of such an obstruction, and said guidewire used to probe the obstruction. If said guidewire is able to enter into an obstruction, but then its forward progress is arrested despite customary maneuvers by an operator, then a balloon catheter can be introduced over the guide wire as far as it can reasonably go without its distal tip going past said guidewire. Then, said balloon is inflated by an operator, creating a zone of blunt dissection or separation at the balloon and extending proximally and distally for a distance of several millimeters. Because of that, the guidewire can be liberated and able to be advanced again, either with said balloon still inflated or after deflation of said balloon. The guidewire may then be able to be passed entirely through an obstructing lesion to enter into a patent segment of the blood vessel, or it may become obstructed again further down field. If the guidewire becomes stuck again, the balloon can be deflated, which results in re-wrapping of the balloon, and the balloon catheter advanced again as far as it can without exceeding the guidewire. The balloon can be inflated again in that new location, and in this way an operator can serially march down an obstructing lesion to eventually cross it in its entirety and enter into a patent blood vessel segment. At that point, other interventions can be done, such as for example atherectomy or definitive angioplasty with balloon catheters, among others.

In the case where an injectable balloon catheter is used to perform these maneuvers, the operator is at liberty to inject radiopaque contrast at any point during the lesion traversal to check their progress, to assess any dissection or vessel perforation, to try to visualize the best path to pursue with the guidewire, and ultimately, to confirm proper intravascular location when the obstructing lesion is completely crossed. For that final element, exit of radiopaque contrast material out of a distal endhole is desirable. In order to accomplish that, in some embodiments of an injectable balloon catheter a guidewire may be retracted proximal to a balloon into a shared injection/guidewire lumen, said shared injection/guidewire lumen being oversized to a guidewire proximal to said balloon, so that said guidewire does not serve to obstruct and act as a valve or narrowing at said catheter's distal tip or distal shaft, therefore injected fluid is able to exit said distal endhole and, in the case of radiopaque contrast, to opacify or allow visualization of distal runoff vessels distal to said obstruction.

It will be known to those familiar with the art that lower profile balloons of smaller diameter may be desired for lesion traversal maneuvers, but once an obstructing lesion is traversed replacement by catheters with larger diameter balloons may be desirable to achieve the optimal result in terms of post-dilatation vessel inner diameter and blood flow.

One exemplary embodiment of the invention uses a balloon catheter that comprises injectability features to allow an operator to observe progress using fluoroscopy by injection of radiopaque contrast agents. Such injectable balloon catheter configurations may be similar to those described in U.S. application Ser. No. 18/106,262 (Murphy), U.S. application Ser. No. 19/384,183 (Murphy), or U.S. Pat. No. 8,532,749 (Patton). An injectable balloon catheter may comprise injection exit ports proximal to a balloon, distal to a balloon, or both, and permit injection while in an over-the-wire configuration either through oversizing of a shared guidewire/injection lumen or through a dedicated injection lumen. If an oversized shared guidewire/injection lumen is used for injection, often a valve is configured at or proximal to a distal tip that effective blocks exit of injected fluids from a distal tip and directs injected fluid out of an exit port through a catheter sidewall.

Another exemplary embodiment of an injectable balloon catheter suitable for use in the invention comprises a biaxial-coaxial transition comprises a hub end and a distal tip, between which there is a tubular shaft, upon said tubular shaft there is an expansile member such as an inflatable balloon generally disposed toward said distal tip of said shaft, and a hub adapter at said hub end comprising two injection ports, said two injection ports being in continuity with at least two lumens within said shaft, said at least two lumens comprising at least a first lumen for passage of a guide wire and injection of fluids therethrough, and at least a second lumen as a means for inflation of said expansile member. A hub apparatus may combine one or more openings often comprising Luer connectors, a manifold for communication of openings with inner lumens of a catheter, strain relief to reduce stresses in the hub to catheter connection, among other elements. In a preferred embodiment, said injection of fluids occurs by an operator attaching a syringe with fluid to a hub connector, and injecting said fluid into said first lumen, said fluid passing down shared guidewire lumen with or without a guidewire therethrough and exiting said first lumen through at least one aperture through a sidewall of said tubular shaft such that said fluid then enters into the body of a patient, for example in a blood vessel. Said injection can also be done in some examples using a power injector.

In one exemplary embodiment, a proximal shaft comprises at least two lumens that are substantially biaxially oriented in relation to each other, and at least a distal shaft comprising at least two lumens that are substantially coaxially oriented in relation to each other. In some embodiments there is a transition in said shaft between said proximal shaft and said distal shaft, said transition comprising an exchange wherein said at least two lumens transform said orientation in relation to each other from substantially biaxial to substantially coaxial.

In order to place injected fluid closest to an area of interest, an injectable balloon catheter would comprise an aperture as distal as possible, and therefore a transition between a proximal biaxial catheter segment and a distal coaxial catheter segment would be closer to a distal tip than a hub end of a catheter. In another exemplary embodiment, said aperture would be located distally within said proximal shaft, proximal to said transition, for example between 1 mm and 40 cm proximal to said transition.

In an exemplary embodiment of an injectable balloon catheter said distal coaxial catheter segment would comprise an inner tube and an outer tube, said inner tube accommodating a guidewire by having an I.D. within 0.001″ to 0.003″ of a recommended guidewire O.D. and be uniform in I.D. without any tapering or expansion, and be rigid and not capable of collapse when said guidewire is removed. Since said I.D. of said distal coaxial catheter segment inner tube is not much larger than said recommended guidewire said guidewire when in situ throughout an entire length of said inner tube would comprise a flow restricting valve that would direct injected fluid in a proximal catheter segment away from an inner lumen of said inner tube and out through said aperture.

In some embodiments of an injectable balloon catheter, said transition comprises a taper between said proximal shaft and said distal shaft, said transition having a first larger outer diameter (O.D.) proximally and a second smaller O.D. distally. In one embodiment of the invention, said proximal shaft has an O.D. of substantially between 4 and 5 French, or between 4.5 and 5 French, and said distal shaft has an O.D. of substantially between 2 and 3 French, or between 2.5 and 3 French, or between 2 and 4 French. In such an embodiment, said proximal shaft would be at least 0.5 French sizes larger than said distal shaft, or up to 3 French sizes larger, said taper comprising a reduction of between 0.5 French (0.167 mm diameter) and 3 French (1 mm diameter) over a length of said tubular shaft ranging between 0.5 mm and 20 cm.

In one embodiment of an injectable balloon catheter, said transition comprises a length of between 1 mm and 75 cm, or between 1 mm and 30 mm. Those familiar with the art will appreciate that other embodiments of the invention will comprise shaft diameters in the typical range known in the art, for example, between 2 French and 10 French. In one embodiment of the invention, said transition is disposed more distally than proximally, however, those familiar with the art will appreciate that a transition may be located anywhere between said hub end and said balloon. Furthermore, those familiar with the art will readily appreciate that said at least two lumens can have many different cross-sectional shapes, for example, a biaxial configuration may comprise lumens with cross-sections that are generally circular or not circular. For example, biaxial lumens can have cross-sections that are generally ovals, semi-circles or crescents, and oval, semi-circular, or crescent cross-section configurations can offer functional advantages as readily known to those familiar with the art. Those familiar with the art will further appreciate that a transition from biaxial to coaxial configuration may entail thinning of a wall of a lumen, for example, said biaxial lumens may have thicker walls than said coaxial lumens, in an exemplary embodiment.

Those familiar with the art will appreciate that advantages of an balloon catheter that comprises a transition between a biaxial and coaxial segment including having a relatively higher profile proximal biaxial segment thereby imparting superior pushability plus a lower-profile distal coaxial segment having improved trackability compared with a biaxial proximal segment, thereby facilitating angioplasty of a small artery, for example a tibial artery or a coronary artery.

Those familiar with the art will appreciate that the transition between a biaxial and coaxial lumen configuration is facilitated by a reduction in the caliber of the outer diameter (O.D.) of the catheter shaft comprising reduction of the inner diameter (I.D.) and O.D. of said first lumen from said biaxial segment, said reduction of I.D. and O.D. of said biaxial segment facilitating envelopment of said first lumen by said second lumen in the transition.

Moreover, those familiar with the art will readily appreciate that use of an injectable balloon catheter for balloon-assisted crossing of blood vessel obstructions that comprises an embodiment that uses a shared guidewire/injection lumen will further comprise a first lumen that is oversized for said guide wire, thereby permitting use of a portion of said first lumen for injection of fluids while said guide wire remains in place in said first lumen, said fluids flowing alongside said guide wire, and if an port is incorporated into a sidewall of a distal aspect of said biaxial lumen, and said first lumen narrows to approximate an outer diameter of said guidewire distal to said wherein said balloon catheter comprises a means for injection of medical fluids that exit said balloon catheter proximal to said balloon thereby entering into the body of a patient while said guidewire resides throughout an entire lumen of said balloon catheter between a proximal end and a distal end.

Such a configuration would comprise a method for performance of a contrast angiography through said first lumen while said guide wire remained in place within said first lumen. A size mismatch between said first lumen I.D. and said guidewire O.D. would have to be sufficient as a means to permit clinically useful injection rates, e.g., at least 0.5 ml/second of iodinated contrast at 300 mg/ml iodine concentration by hand injection or power injector at 300 psi pressure. In one exemplary embodiment an injectable balloon catheter with a biaxial-coaxial transition comprises a distal coaxial segment compatible with a 0.014″ guidewire, thereby requiring a minimum inner diameter of 0.015″, 0.016″, or greater, whereas a proximal biaxial segment comprises a minimum inner diameter at least 0.002″ larger, for example between 0.002″ and 0.062″ larger, to allow clinically useful flow rates of injected fluids to be achieved by hand injection or power injection.

Similarly, in an exemplary embodiment of an injectable balloon catheter a cross-sectional area of said first lumen in said coaxial segment would be smaller than a cross-sectional area in said biaxial segment, for example by at least 0.25 mm², or between 0.25 mm² and 0.75 mm².

In an exemplary embodiment of an injectable balloon catheter, a biaxial segment is located proximal to a coaxial segment, that is, closer to a hub and operator. The lengths can be adjusted by experimentation to suit clinical needs, and a proximal biaxial segment may be longer or shorter than a distal coaxial segment. The distal coaxial segment would comprise a balloon, and in one embodiment a distal coaxial segment would comprise at least one millimeter of length proximal to an inflatable element or balloon, either comprising an aspect of an balloon proximal neck or even proximal to said balloon proximal neck, but could be much longer, such as for example between one millimeter and 3 cm, or between one millimeter and 20 cm, or between one millimeter and 100 cm.

The foregoing ranges are set forth solely for the purpose of illustrating typical device dimensions. The actual dimensions of a device constructed according to the principles of the present invention may obviously vary outside of the listed ranges without departing from its basic principles.

An injectable balloon catheter also requires a means of injection of medical fluids into a shared guidewire-injection lumen that comprises a means of preventing backflow of fluids injected thereto, which would result in fluid leaking out of the hub end and external to the catheter. A means of preventing backflow could be a choke or diameter reduction in the proximal biaxial segment guidewire-injection lumen, or alternative an integral or modular restrictor within or attached to the catheter hub such that a difference between an outer diameter of a guidewire residing through said first lumen and an inner diameter of said choke would be within 0.010″, preferably between 0.001 and 0.010″.

### BRIEF DESCRIPTION OF THE DRAWINGS

FIG. 1 depicts a preferred embodiment of an injectable balloon catheter with said balloon in collapsed configuration as seen from a lateral surface rendering view.

FIG. 2 depicts a preferred embodiment of an injectable balloon catheter with said balloon in expanded configuration as seen from a lateral surface rendering view.

FIGS. 3A through 3H are lateral cutaway renderings depicting sequential combined advancement of a balloon catheter and guidewire across a blood vessel obstruction.

FIGS. 4A and 4B are lateral cutaway views of a distal portion of a catheter to illustrate an embodiment of the invention. FIG. 4A shows a guidewire extending beyond a distal catheter tip whereas FIG. 4B shows a guidewire whose distal tip terminates within a catheter.

### DETAILED DESCRIPTION

Certain exemplary embodiments will now be described to provide an overall understanding of the principles of the structure, function, manufacture, and use of the methods and devices disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Devices and related methods are disclosed that generally comprise a method of crossing a blood vessel obstruction using a combination of a balloon catheter and a guidewire.

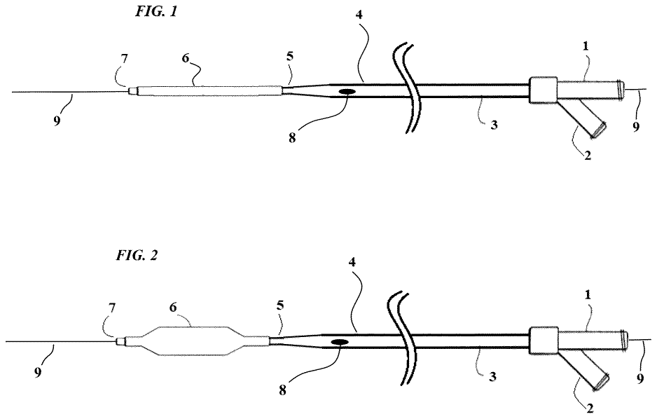

FIG. 1 depicts an exemplary embodiment of an injectable balloon catheter with a proximal hub end comprising a first port 1 for passage of a guide wire 9 and injection of fluids there through, a second port 2 as a means for injection or aspiration of fluid or gas as means of inflation or deflation of an inflatable balloon 6 generally disposed toward a distal tip 7 of said balloon catheter, said balloon 6 in this illustration in collapsed configuration, said balloon catheter further comprising a shaft which is a substantially tubular element generally disposed between said proximal hub end and said distal tip 7, said shaft comprising at least two lumens, including at least a first lumen for passage of a guide wire and injection of fluids there through, and at least a second lumen for injection of fluids or gases as a means of inflation of said balloon 6, said second lumen in fluid communication with an interior of said balloon 6, in this preferred embodiment said shaft comprising a proximal shaft 3 which comprises two lumens oriented in a biaxial configuration in which said two lumens are substantially parallel and side-by-side, said shaft further comprising a distal shaft 5 which comprises two lumens oriented in a coaxial configuration in which one lumen is contained in and circumscribed by the other lumen, in this preferred embodiment said shaft further comprising a transition 4 comprising a location for transformation of the orientation of the two lumens from a biaxial configuration to a coaxial configuration. In this exemplary embodiment, said transition 4 further comprises a taper, wherein said proximal shaft has a larger O.D. than said distal shaft, for example, by between 0.5 and 4 French sizes. FIG. 1 further provides an exemplary illustration of an aperture that comprises an injection exit hole 8, whereby fluids injected into a shared guidewire-injection lumen are able to exit the catheter and into a patient's body, such as for example into an interior of a blood vessel of a patient.

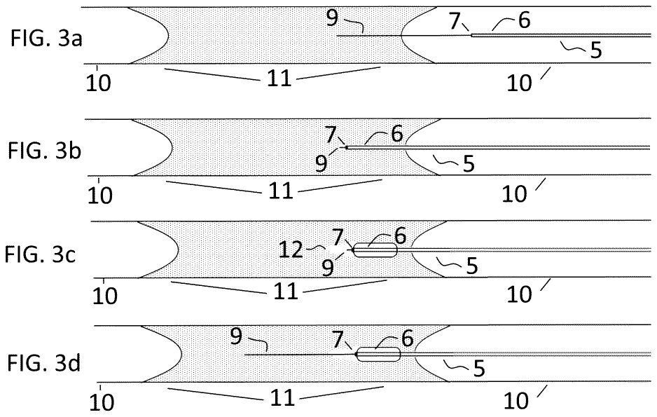

FIG. 2 is an exemplary embodiment of an injectable balloon catheter but in contrast to FIG. 1 in this illustration said balloon 6 is depicted in its inflated configuration.

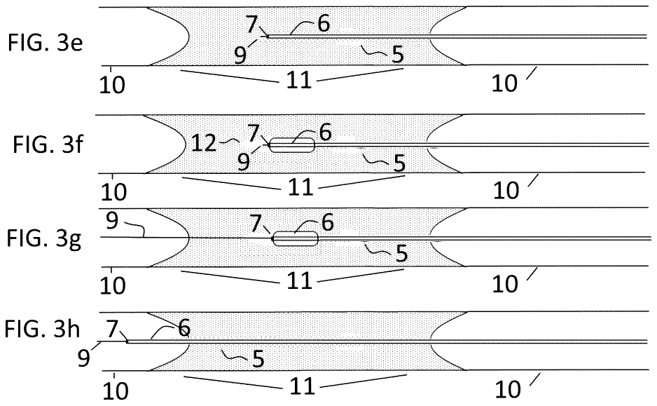

FIG. 3A-H are a series of illustrations that depict an exemplary embodiment of the invention by showing a sequence of steps that together comprise balloon catheter-assisted traversal of an exemplary blood vessel obstruction, depicted in longitudinal cutaway view. In FIG. 3A nonobstructed blood vessel segments 10 surround an obstruction 11 shown with hash marks. A distal shaft 5 with a balloon 6 in collapsed configuration and a low-profile distal tip 7 is passed over a guidewire 9 that enters from a patent blood vessel segment 10 into an obstruction 11. Said guidewire 9 has become obstructed in a proximal aspect of said obstruction 11 and cannot be advanced by an operator any further. FIG. 3B shows advancement of said distal shaft 5 over said guidewire 9 into said obstruction, with said tip 7 close to a distal guidewire 9 end. In FIG. 3C said balloon 6 is inflated, creating a zone of separation 12 within said obstruction 11. The zone of separation allows further advancement of said guidewire 9 in FIG. 3D, further into said obstruction 11 but not through it into said non-obstructed blood vessel segment 10.

FIG. 3E repeats the sequence; after deflation of said balloon 6 said distal shaft 5 is advanced further along said guidewire 9 so again said catheter tip 7 is near a distal end of said guidewire 9. Again said balloon 6 is inflated in FIG. 3F, creating another zone of separation 12, thereby permitting further advancement of said guidewire 9 in FIG. 3G, this time through a distal aspect of said obstruction 11 and into a nonobstructed blood vessel segment 10. After deflation of said balloon 6, said catheter can be advanced over said guidewire in FIG. 3H. After that, other procedures can be done to relieve said blood vessel obstruction at the discretion of an operator.

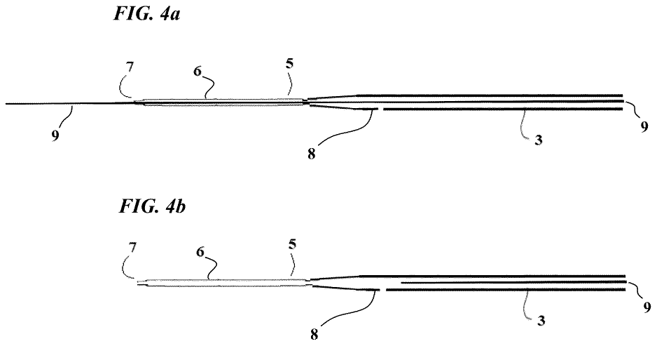

FIG. 4A is a lateral cutaway view of a distal portion of an injectable balloon catheter with an exit hole 8 through a sidewall proximal to a balloon 6, also depicting a guidewire 9 in situ, through and through, extending out of the distal tip 7 of the catheter. FIG. 4B shows a lateral cutaway view of a distal portion of an injectable balloon catheter with a injection exit hole 8 through a sidewall proximal to a balloon 6, but in this case depicting a guidewire located so that it does not extend out of a distal tip 7 of the catheter but rather ends distally in a proximal shaft 3. In both FIGS. 4A and 4B the catheter proximally and the catheter hub components are not shown. One familiar with the art will understand that if configured and sized properly, said guidewire 9 as shown in FIG. 4A would serve as a valve when positioned through said distal tip, if within one or two thousandths of an inch in OD compared to an ID of said distal shaft 5 or said distal tip 7, so that when fluids are injected proximally they will flow down the catheter and exit almost exclusively through said injection exit hole 8 through a sidewall of said catheter, with a clinically insignificant amount exiting said catheter distal tip 7. However, when said guidewire 9 is configured to end proximally within said proximal shaft 3, said proximal shaft 3 having a substantially larger cross-sectional area than said guidewire 9, for example at least 0.25 mm² larger or between 0.25 mm² and 0.75 mm² larger, then when fluid is injected proximally it will flow through the catheter and exit from both said injection exit hole 8 through a side wall of a catheter as well as through said catheter distal tip 7, both in clinically useful amounts. One application of this would be that if an operator wanted to inject contrast into a blood vessel proximal to said distal tip 7 and proximal to said balloon 6, said guidewire 9 would be positioned as show in FIG. 4A extending out of said distal tip 7, whereas if an operator desired to visualize forward or distal of said catheter distal tip 7 then the operator would retract or position said guidewire within said oversized proximal shaft, so it does not obturate said narrower distal shaft 5, and when contrast is injected with said guidewire 9 in such a proximal position within said proximal catheter shaft 3 as shown in FIG. 4B, contrast would exit both said injection exit hole 8 and said distal tip 7 in clinically useful amounts, allowing visualization of blood vessels beyond said distal tip 7. That can be useful because it can help an operator understand whether they are in a proper lumen that can be revascularized using catheter techniques and in the case of an artery allow distal perfusion through downstream non-obstructed arteries, versus being in an unsuitable position for a catheter revascularization procedure, such as within a wall of an artery or perforated to an extravascular space outside of an artery.

I claim:

1. A method of facilitating traversal of a blood vessel obstruction, comprising positioning a balloon of a balloon catheter within an obstruction and inflating said balloon within said obstruction to create space within said obstruction and advancing a guidewire further through said obstruction following balloon inflation.

2. The method of claim 1, wherein the balloon is intermittently inflated and deflated within the obstruction.

3. A method for performing a medical procedure comprising a trans-catheter traversal of a blood vessel obstruction, comprising:
a) gaining catheter and guidewire access into a blood vessel lumen,
b) advancing a guidewire into a blood vessel obstruction until it cannot be advanced further,
c) advancing a balloon catheter over said guidewire and into said blood vessel obstruction,
d) inflating said balloon,
e) advancing said guidewire further into said blood vessel obstruction,
f) deflating said balloon,
g) advancing said catheter,
h) repeating steps b through g until said blood vessel obstruction is traversed and said guidewire enters into a nonobstructed blood vessel beyond said obstruction.

4. The method for performing a medical procedure of claim 3, wherein step (e) occurs after step (f).

5. The method of performing a medical procedure of claim 3 wherein said balloon catheter comprises an injection lumen and at least one exit hole configured for injection of medical fluids to exit said balloon catheter proximal to said balloon thereby entering into the body of a patient while said guidewire resides throughout an entire lumen of said balloon catheter between a proximal end and a distal end.

6. The method of performing a medical procedure of claim 3 wherein said balloon catheter comprises an injection lumen and at least one exit hole configured for injection of medical fluids to exit said balloon catheter from a catheter distal tip thereby entering into the body of a patient while said guidewire extends within a proximal shaft of said balloon catheter and terminates proximal to said distal tip.

7. The method of claim 1, wherein the balloon is intermittently inflated and deflated while advancing the guidewire.

8. A catheter system comprising:
a. a guidewire; and
b. means for modifying a blood vessel obstruction to facilitate advancement of the guidewire,
wherein the means comprises:
c. an inflatable balloon mounted on a shaft;
d. a first lumen configured to receive the guidewire;
e. a second lumen configured to deliver inflation fluid; and
f. a proximal shaft having a biaxial lumen configuration and a distal shaft having a coaxial lumen configuration.
